(19) United States
(12) Patent Application Publication
(10) Pub. No.: US 2012/0123466 A1
(43) Pub. Date: May 17, 2012

(54) AXIALLY VARIABLE RADIAL PRESSURE CAGES FOR CLOT CAPTURE

(75) Inventors: Stephen C. Porter, Oakland, CA (US); James B. Kellett, Los Gatos, CA (US); Del Kjos, Pleasanton, CA (US)

(73) Assignees: STRYKER NV OPERATIONS, LTD., Dublin (IE); STRYKER CORPORATION, Kalamazoo, MI (US)

(21) Appl. No.: 13/291,749

(22) Filed: Nov. 8, 2011

Related U.S. Application Data
(60) Provisional application No. 61/413,174, filed on Nov. 12, 2010.

(51) Int. Cl. A61F 2/00

(52) U.S. Cl. 606/200

(57) ABSTRACT

A device for removing a blood clot from a lumen of a vessel, the device comprising a pusher and an expandable tubular cage fixedly engaged to the pusher. The tubular cage has a proximal end, a distal end, and a wall extending therebetween. The wall comprises a plurality of bands of cells axially arranged along the tubular cage, wherein one band of cells comprises at least one skiving cell having a cell wall with a proximal portion, a distal portion, and a central portion between the proximal portion and the distal portion. The central portion deforms radially inward in response to a radially applied force to a greater extent than the distal portion.

### CROSS REFERENCE TO RELATED APPLICATIONS

The present application claims the benefit under 35 U.S.C. §119 to U.S. provisional patent application Ser. No. 61/413,174, filed Nov. 12, 2010. The foregoing application is hereby incorporated by reference into the present application in its entirety.

### BACKGROUND

Thrombectomy cages are used to treat certain conditions, such as strokes where blood flow in a vessel is blocked by the narrowing of the vessel or the formation of a blood clot. These devices function to remove a blood clot and recanulate the vessel lumen by compressing the clot into the lumen wall, macerating the clot by pulling the device through the clot, capturing the clot by pulling the clot into the interior of the device, breaking the clot into smaller pieces to facilitate aspiration, anchoring the clot so that it does not migrate distally during aspiration, and combinations thereof.

Prior art devices (such as those described in U.S. Patent Publication Nos. 2002/0058904 and 2007/0208367, incorporated herein by reference in their entireties) use a large radial force to tear through the clot as the device expands. After the clot has been torn by the device, the clot penetrates into the interior of the device to be captured in a dense net at the distal end of the device. In such devices, the pressure needed to sever the fibrin networks of the blood clot is high. Other prior art devices have “skived” the clot (where “skiving” is defined as cutting or tearing the clot from the wall of the vessel using a shear force), where an axial force is applied to the device rather than radial forces to tear the clot from the wall of the vessel.

### SUMMARY

In accordance with various embodiments of the invention, a device for removing a blood clot from a lumen of a vessel comprises a pusher and an expandable tubular cage fixedly engaged to the pusher. The tubular cage has a proximal end, a distal end, and a wall extending therebetween. The wall comprises a plurality of circumferential bands of cells axially arranged along the tubular cage, wherein one band of cells comprises at least one skiving cell having a cell wall with a proximal portion, a distal portion, and a central portion between the proximal portion and the distal portion. The central portion preferably deforms radially inward in response to a radially applied force to a greater extent than the distal portion.

In at least one embodiment, the deformation of the central portion is at least about 25% more than the deformation of the distal portion. In at least one embodiment, the deformation of the central portion is at least about 30% more than the deformation of the distal portion.

In at least one embodiment, the distal portion of the skiving cell is stiffer than at least the central portion. In at least one embodiment, the distal portion is thicker than at least the central portion. In at least one embodiment, the distal portion is wider than at least the central portion. In at least one embodiment, a distal angle of the distal portion is greater than a proximal angle of the proximal portion. In at least one embodiment, the proximal portion and the distal portion are thinner than the central portion. In at least one embodiment, an axial length of the central portion is at least about 0.5 times a diameter of the vessel wall.

In at least one embodiment, the device lacks any mechanism for detachment of the expandable tubular cage from the pusher. In at least one embodiment, the wall is formed of a structural material arranged in a single layer such that there are no material crossover points anywhere along the wall.

In at least one embodiment, a device for removing a blood clot from a vessel wall, the device comprising a pusher and an expandable tubular cage fixedly engaged to the pusher. In at least one embodiment, the tubular cage has a proximal end, a distal end, and a wall extending therebetween. The wall is formed of a plurality of cells defining openings in the wall of the cage. In at least one embodiment, the wall comprises a proximal end region at the proximal end of the cage; a distal end region at the distal end of the cage; and at least one intermediate region therebetween. At least one cell of the intermediate region is a skiving cell having a cell wall with a proximal portion, a distal portion, and a central portion between the proximal portion and the distal portion. In at least one embodiment, the central portion deforms radially inward in response to a radially applied force to a greater extent than the distal portion.

In at least one embodiment, an axial length of the central portion is at least about 0.5 times a diameter of the vessel wall.

In at least one embodiment, the deformation of the central portion is at least about 25% more than the deformation of the distal portion. In at least one embodiment, the deformation of the central portion is at least about 30% more than the deformation of the distal portion.

In at least one embodiment, the at least one intermediate region has a first band of skiving cells defines first openings and a second band of cells defines second openings, where the first openings are greater than the second openings.

In at least one embodiment, the intermediate region comprises at least one circumferential band of skiving cells having cell walls defined by a proximal strut pair and a distal strut pair; and an adjacent circumferential band of cells having a proximal strut pair, a distal strut pair, and a divider strut connects a first strut of the proximal strut pair to a second strut of the distal strut pair.

In at least one embodiment, a first intermediate region has at least one band of skiving cells and an axially adjacent circumferential band of cells has a greater cellular density than the band of skiving cells.

In at least one embodiment, a first intermediate region has at least one band of skiving cells and a second intermediate region has a plurality of bands of cells, wherein a cellular density of the second intermediate region is greater than a cellular density of the first intermediate region.

In at least one embodiment, the cell wall of the skiving cell comprises a proximal strut pair, a central strut pair, and a distal strut pair.

### BRIEF DESCRIPTION OF THE DRAWINGS

FIG. 1 shows a prior art cage deployed in a lumen of a vessel that has a clot attached to the vessel wall.

FIG. 2 shows an embodiment of the cage of the present invention having a plurality of non-uniform openings and deployed in a lumen of a vessel, where the vessel has a clot attached to the vessel wall.

FIG. 3 shows a perspective view of an embodiment of the cage of the present invention.

FIG. 4 shows a flat view of the embodiment of the cage shown in FIG. 3.

FIGS. 5A-5C show flat views of embodiments of the cage.

FIGS. 6A-6C show flat views of embodiments of the cage, with progressively less cell density in the intermediate region 152 of the cage 100.

FIG. 7 shows a flat view of an embodiment of the cage.

FIG. 8 shows a flat view of an embodiment of the cage.

FIGS. 9A-9D show flat views of embodiments of the cage.

FIG. 10A shows a plan view of an embodiment of the cage. FIGS. 10B-10D show flat views of embodiments of the cage shown in FIG. 10A.

FIG. 11A shows a plan view of an embodiment of the cage. FIGS. 11B-11D show flat views of embodiments of the cage shown in FIG. 11A.

### DETAILED DESCRIPTION OF ILLUSTRATIVE EMBODIMENTS

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

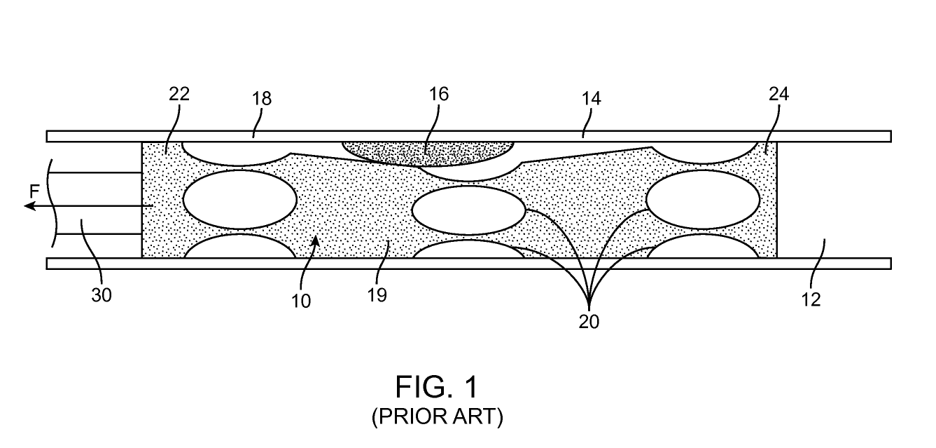

FIG. 1 shows a prior art cage 10 deployed in a lumen 12 of a vessel 14 having a blood clot 16 attached to a wall 18 of the vessel 14. Cage 10 has a wall 19 that defines uniform openings 20 along the entire length of the cage 10 that allows for uniform deployment of the cage from proximal end 22 to distal end 24. Proximal end 22 is connected to an end of a pusher 130. As shown in FIG. 1, only a small portion of the cage nearest the ends 22, 24 contacts the wall 18 that has the blood clot 16. An axial force F is applied to the cage 10, but only a small portion of the cage 10 is used to skive the blood clot 16.

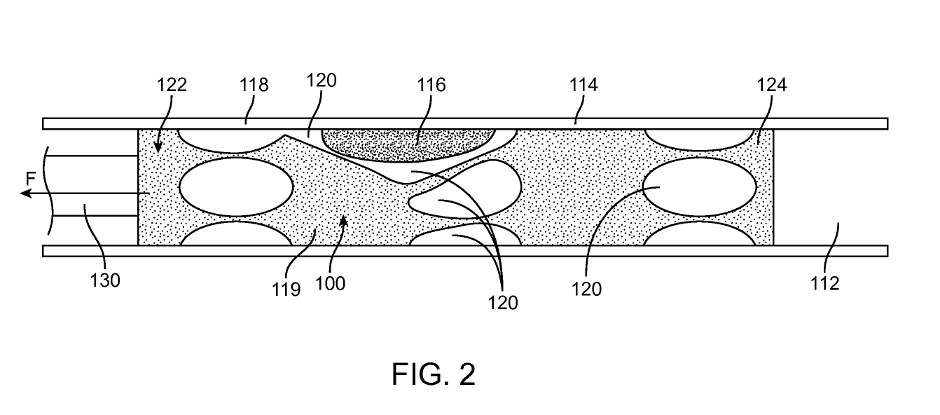

FIG. 2 shows schematically a cage 100 of the present invention deployed in a lumen 112 of a vessel 114 having a blood clot 116 attached to a wall 118 of the vessel 114. Cage 100 has a wall 119 with a plurality of non-uniform openings 120. At least one of the openings engages with the blood clot 116 more favorably than the opening 20 of the prior art cage 10 shown in FIG. 1. Cage 100 extends from a proximal end 122 towards a distal end 124. The proximal end is connected to a pusher 130.

In at least one embodiment, at least one skiving cell has an opening 120 defined by a cell wall having proximally weaker and distally stronger portions such that the cell wall deforms radially inward near a central portion of the cell wall in response to a radially applied force to a greater extent than the distal portion of the cell wall. The radially applied force can, in some instances, occur when the cage contacts the clot. The radially applied force can also be a uniformly applied force, such as an expansive force. Other radial forces applied to the cage can cause the central portion of the cell wall to deform radially inward to a greater extent than the distal portion of the cell wall. In some embodiments, the deformation of the central portion radially inward is at least about 25% more than the deformation of the distal portion. In some embodiments, the deformation of the central portion radially inward is at least about 30% more than the deformation of the distal portion.

Because cage 100 deforms in this manner, an opening 120 of a skiving cell is able to present itself more favorably to engage with the blood clot 116 while the remainder of the cage 100 contacts a greater portion of the vessel wall 118 than the prior art cage shown in FIG. 1. This increased contact area (as well as the stronger distal end in at least some of the openings 120) results in improved skiving of the clot to sever the fibrin network and trap the clot into the cage when the axial force F is applied.

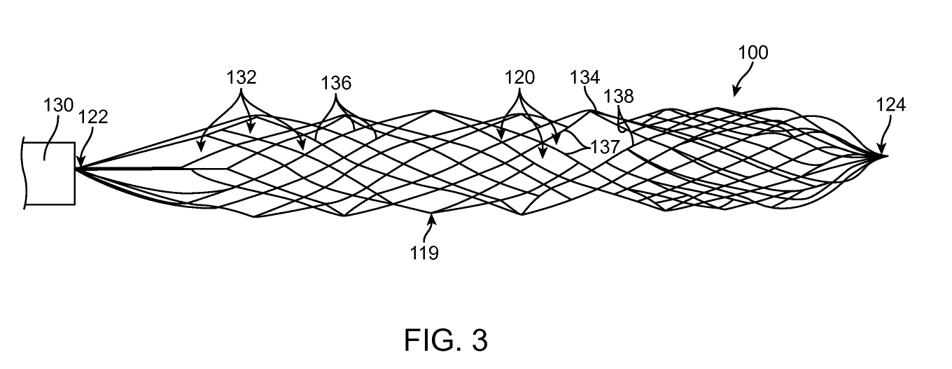

FIG. 3 shows an embodiment of a device of the present invention in an expanded state, including cage 100 and pusher 130. In some embodiments, such as the one shown in FIG. 3, the cage 100 is closed at both the proximal end 122 and the distal end 124 in the expanded state. In other embodiments, the cage 100 is only closed at one end. In one embodiment, the cage 100 is open at both the proximal end 122 and the distal end 124. As shown in FIG. 3, the proximal end 122 is connected to a distal end of the pusher 130. In some embodiments, the proximal end 122 and the distal end 124 are connected to the pusher 130. Other configurations of attaching the cage 100 to the pusher 130 are within the scope of this invention. In some embodiments, the device lacks any mechanism for detaching the cage 100 from the pusher 130. Thus, in such embodiments, the cage 100 is removed from the vessel with the pusher 130 still attached.

In some embodiments, such as the one shown in FIG. 3, cage 100 has a plurality of circumferential bands of cells 132 that form wall 119 of the cage. Each cell 132 is formed by a cell wall 134 having a proximal portion, a central portion, and a distal portion. Each cell wall 134 is formed by a plurality of struts 136. In at least the embodiment shown, the cell wall 134 has a proximal strut pair 137 and a distal strut pair 138. The cell wall 134 defines an opening 120 in the wall 119 of the cage. In at least one of the cells, the central portion of the cell deforms radially inward in response to a radially applied force to a greater extent than the distal portion. Because of this deformation in the cell wall 134 of the at least one skiving cell, in some embodiments the cage 100 has a non-uniform diameter along at least a portion of its length between a proximal end and distal end. In at least one embodiment, an axial length L of the central portion of the skiving cell is at least about 0.5D, where D is the diameter of the vessel to be treated. In some embodiments, L is at least about 0.75D. In some embodiments L is about 1.0D. In some embodiments, L is between about 0.5D and about 3.0D.

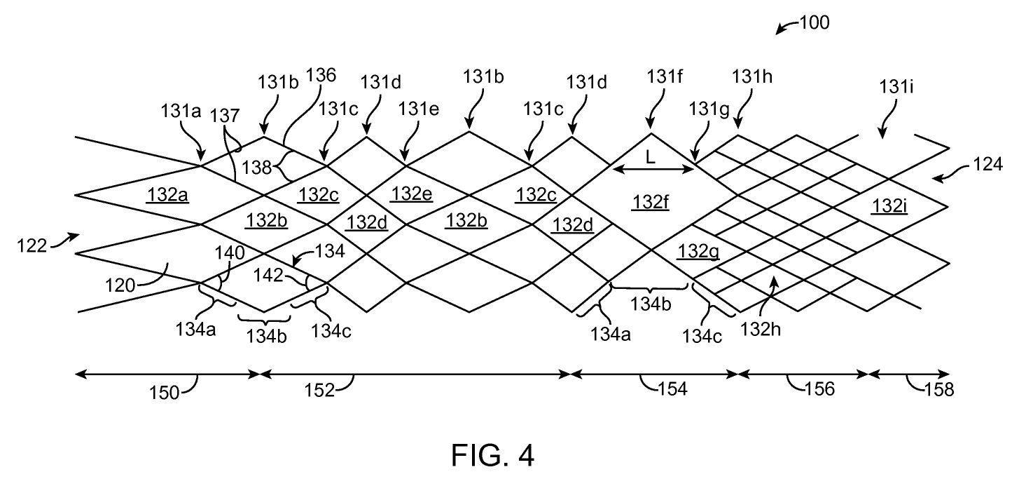

FIG. 4 shows a flat view of the cage 100 of FIG. 3 having a plurality of circumferential bands 131 of cells 132. Each cell is formed by a cell wall 134 having a proximal portion 134a, a central portion 134b, and a distal portion 134c. Each cell wall 134 is formed by a plurality of struts 136. In at least the embodiment shown, the cell wall 134 has a proximal strut pair 137 and a distal strut pair 138. The proximal strut pair 137 has a proximal apex angle 140, and the distal strut pair has a distal apex angle 142.

These cells 132 are arranged into a proximal end region 150 at the proximal end 122 of the cage, a first intermediate region 152, a second intermediate region 154, a third intermediate region 156, and a distal end region 158 at the distal end of the cage. The proximal end region 150 is connected to the first intermediate region 152, which is connected to the second intermediate region 154, which is connected to the third intermediate region 156, which is connected to the distal end region 158. Each region 150, 152, 154, 156, 158 has at least one circumferential band 131 of cells 132.

In the embodiment shown in FIG. 4, each one of these regions 150, 152, 154, 156, 158 has cells 132 with different structures relative to an adjacent region, which creates a non-uniform pattern of cells 132 (and therefore a plurality of non-uniform openings) along the length of the cage 100. In some embodiments, this non-uniform pattern of cells 132 (therefore defining a non-uniform pattern of openings 120) allows the cage 100 to have cells 132 of differing radial strengths throughout the cage 100 such that at least one opening is able to engage with a blood clot in a vessel depending on the size or shape of the blood clot. In some embodiments, the cells 132 are non-uniform in cross-section (by having struts 136 with different widths and/or thicknesses, for example) or non-uniform in size or shape (by having struts 136 with different lengths, for example).

In the embodiment shown in FIG. 4, proximal end region 150 has a circumferential band 131a of cells 132a, where the struts 136 of the proximal strut pair 137 are longer than the struts 136 of the distal strut pair 138.

The first intermediate region 152, which is connected to the proximal end region 150, has a plurality of cells 132b, 132c, 132d, 132e. A circumferential band 131b of cells 132b is axially adjacent to the circumferential band 131a of cells 132a of the proximal end region 150. In the embodiment shown, cell 132b has strut pairs 137, 138 that have struts 136 of equal length. A circumferential band 131c of cells 132c is axially adjacent to the circumferential band 131b of cells 132b. In the embodiment shown, cell 132c has a proximal strut pair 137 with struts 136 that are longer than the struts 136 of the distal pair 138. A band of cells 132d is axially adjacent to the band of cells 132c. In the embodiment shown, cell 132d has walls 137, 138 that have struts 136 of equal length, similar to cell 132b. However, the proximal apex angle 140 and the distal apex angle 142 of cell 132d are larger than the proximal apex angle 140 and the distal apex angle 142 of cell 132b. A circumferential band 131e of cells 132e is axially adjacent to the circumferential band 131d of cells 132d. Cell 132e has a proximal strut pair 137 with struts 136 that are shorter than the struts 136 of the distal strut pair 138. In the cage 100 shown in FIG. 4, the band of cells 132e is axially adjacent to a second circumferential band of cells 132b, which is axially adjacent to a second circumferential band of cells 132c. The second circumferential band of cells 132c is then axially adjacent to a second circumferential band of cells 132d.

The second intermediate region 154 is connected to the first intermediate region 152 by the second band of cells 132d. The second intermediate region 154 has a band of cells 132f. Although any of the cells 132 could conceivably be designed to be a skiving cell, cells 132f are at least one band of skiving cells in the cage 100. Each cell 132f has a cell wall having proximally weaker and distally stronger portions such that the cell wall deforms radially inward near a central portion 134b of the cell wall in response to a radially applied force to a greater extent than the distal portion 134c of the cell wall. Thus, the central portion deforms radially inward in response to a radially applied force to a greater extent than the distal portion. In some embodiments, the deformation of the central portion is at least about 25% more than the deformation of the distal portion. In at least some embodiments, the deformation of the central portion is at least about 30% more than the deformation of the distal portion. As shown in FIG. 4, cell 132f has strut pairs 137, 138 with struts 136 of equal length. Thus, for the cell wall to have proximally weaker and distally stronger portions, the strut pairs have struts with a tapered thickness or width. As discussed above, in at least one embodiment, an axial length L of the central portion of the skiving cell is at least about 0.5D, where D is the diameter of the vessel to be treated. In some embodiments, L is at least about 0.75D. In some embodiments L is about 1.0D. In some embodiments, L is between about 0.5D and about 3.0D.

In at least one embodiment, the proximal strut pair of the skiving cell can be longer or shorter than the distal strut pair of the skiving cell. In at least one embodiment, the central portion of the skiving cell can be thinner or narrower than at least the distal portion. In at least one embodiment, the cellular density of cells adjacent to the distal portion of the skiving cell can be greater than the cellular density of the cells adjacent to the central portion of the skiving cell. In at least one embodiment, the material properties of the central portion of the skiving cell can differ from the material properties of the distal portion of the skiving cell such that the central portion deforms radially inwardly more than the distal portion of the skiving cell.

The third intermediate region 156 is connected to the second intermediate region 154 by the cells 132f. Cell 132g is adjacent to cell 132f and also has strut pairs 137, 138 that have struts 136 of equal length, but is smaller than cell 132f. A plurality of cells 132h are also axially adjacent to cells 132g and 132f. Cells 132h as shown in FIG. 4 are much smaller and more numerous (resulting in an increased density of cells) than any of the other cells 132 in cage 100. These smaller cells and the increased density of the cells near the distal end 124 of the cage 100 allow the cage 100 to retain portions of the blood clot within the cage 100.

The distal end region 158 is connected to the third intermediate region 156 by the cells 132h. At the distal end of the cage 100, cell 132i has strut pairs 137, 138 with struts 136 of equal length.

While in the above description, each of the cells has been generally described based upon their strut length or apex angles, the width and thicknesses of the struts 136 can also vary along the length of cage 100. For example, the cell wall of cell 132b has a proximal strut pair 137 that is thinner or narrower than the distal strut pair 138. Varying the thicknesses and widths of the struts 136 of the cells 132 will also create a non-uniform cell pattern in the cage 100. In some embodiments, struts 136 can be tapered such that they are wider or thicker at the distal end of the cell 132 than at the central portion of the cell wall. In some embodiments, struts 136 can be tapered such that they are wider or thicker at the proximal end of the cell 132 than at the central portion of the cell wall.

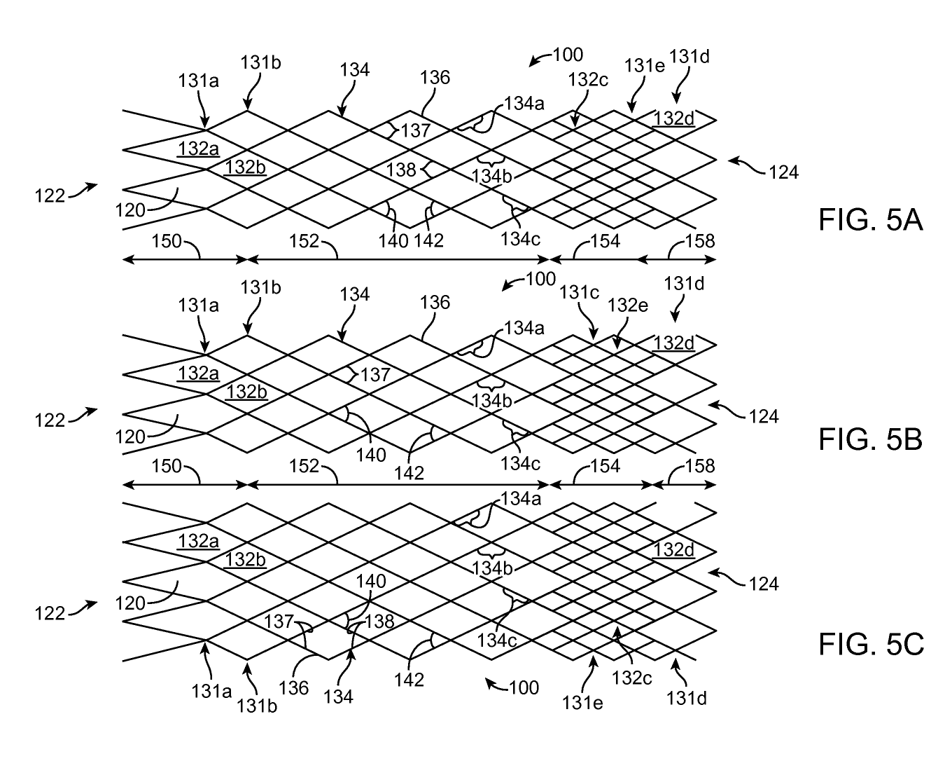

FIGS. 5A-5C show flat patterns of embodiments of the cage 100 with a proximal end region 150 having at least one circumferential band 131a of cells 132a, a first intermediate region 152 having a plurality of circumferential bands 131b of cells 132b, a second intermediate region 154 having a plurality of circumferential bands 131c of cells 132c, and a distal end region 158 having at least one circumferential band 131d of cells 132d at the distal end 124 of the cage. Cells 132a, 132b, 132c, and 132d are non-uniform. In this embodiment, at least some of the cells 132b in the first intermediate region 152 are skiving cells. In this embodiment, at least some of the cells 132c in the second intermediate region 154 retain clot particles within the cage.

Cells 132a have a proximal strut pair 137 and a distal strut pair 138. The struts 136 of the proximal strut pair 137 are longer than the struts 136 of the distal strut pair 138. A plurality of cells 132b are axially adjacent to cell 132a. In this embodiment, at least one of the cells 132b is a skiving cell. Cell 132b has strut pairs 137, 138 that have struts 136 of equal length. However, the struts 136 of proximal strut pair 137 are thinner or narrower than the struts 136 of distal strut pair 137. Thus, a central portion 134b of the cell wall 134 is weaker than at least the distal portion 134c of the cell wall 134.

Cells 132c are axially adjacent to cells 132b. Cells 132c as shown in FIG. 5A are much smaller and denser than any of the other cells 132 in cage 100. These smaller cells and increased density in the cells near the distal end 124 of the cage 100 allows the cage 100 to retain portions of the blood clot within the cage 100.

At the distal end 124 of the cage 100, cell 132d has proximal strut pair 137 with struts 136 of equal length, width, and thickness.

FIG. 5B shows a flat pattern of an embodiment of the cage as shown in FIG. 5A. However, in this embodiment, cells 132b have a proximal strut pair 137 with struts 136 that increase in thickness or width from the proximal end to the distal end of the strut 136. Cells 132b also have a distal strut pair 138 with struts 136 that taper in thickness or width from the proximal end to the distal end of the strut 136.

FIG. 5C shows a flat pattern of an embodiment of the cage as shown in FIG. 5A. However, in this embodiment, only some of the cells 132b have a proximal strut pair 137 with struts 136 that are thinner or narrower than the struts 136 of distal strut pair 138.

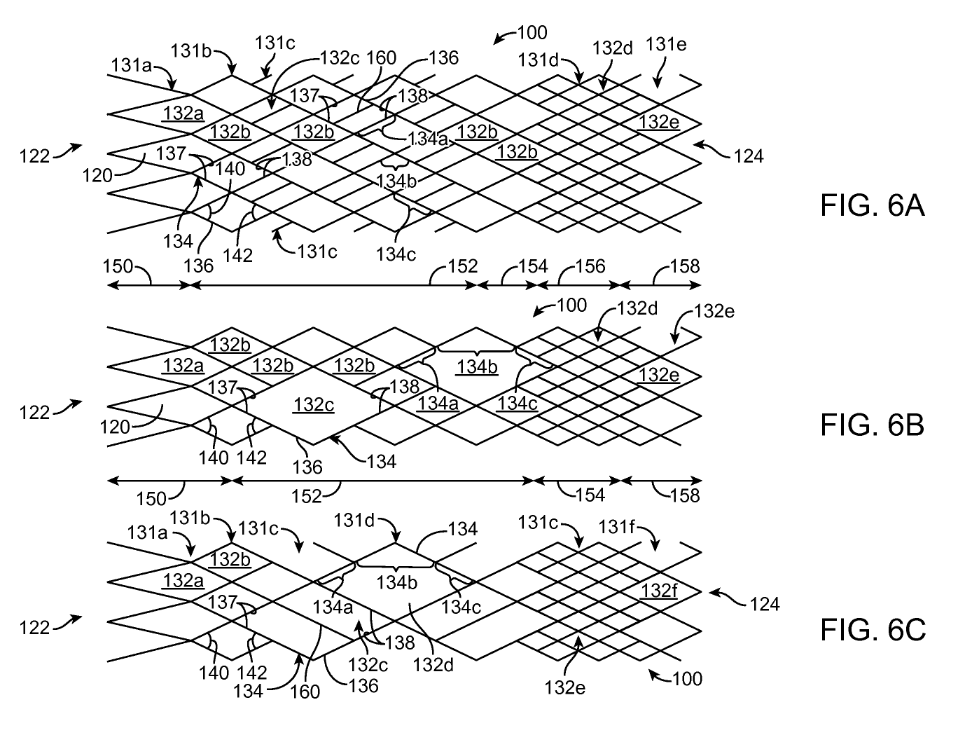

FIGS. 6A-6C show flat views of embodiments for the cage 100 shown in FIG. 4, with progressively less cell density in the intermediate region 152 of the cage 100.

In particular, FIG. 6A shows a cage 100 with a proximal end region 150 at the proximal end 122 of the cage, a first intermediate region 152, a second intermediate region 154, a third intermediate region 156, and a distal end region 158 at the distal end 124 of the cage. The proximal end region 150 has a plurality of cells 132a, where each cell 132a has a proximal strut pair 137 and a distal strut pair 138. The struts 136 of the proximal strut pair 137 are longer than the struts 136 of the distal strut pair 138.

The first intermediate region 152 has a plurality of cells 132b that are axially adjacent to cell 132a, and cell 132b has strut pairs 137, 138 with struts 136 of equal length. Cells 132c are adjacent to cells 132b. Cells 132c have a proximal strut pair 137, a distal strut pair 138, and a divider strut 160 that connects a strut 136 of the proximal strut pair 137 with a strut 136 of the distal strut pair. The cells 132b act as skiving cells where a central portion 134b of the cell wall 134 is weaker than at least the distal portion 134c of the cell wall 134. The distal portion 134c is stronger than the central portion 134b because of the configuration of the surrounding cells 132c, which increase strength near at least the distal portion 134c of the cell wall 134 of cell 132b. Thus, the central portion deforms radially inward in response to a radially applied force to a greater extent than the distal portion. In some embodiments, the deformation of the central portion is at least about 25% more than the deformation of the distal portion. In at least some embodiments, the deformation of the central portion is at least about 30% more than the deformation of the distal portion.

The second intermediate region 154 has a plurality of cells 132b with struts 136 of equal length. In some embodiments, cells 132b in the second intermediate region 154 can also act as skiving cells.

The third intermediate region 156 has a plurality of cells 132e that are much smaller and denser than any of the other cells 150 in cage 100. These smaller cells and increased density in the cells near the distal end 124 of the cage 100 allows the cage 100 to retain clot particles within the cage 100.

The distal end region 158 has a plurality of cells 150f with strut pairs 137, 138 having struts 136 of equal length, width, and thickness.

In FIG. 6B, the cage 100 has a proximal end region 150 at the proximal end 122 of the cage, a first intermediate region 152, a second intermediate region 154, and a distal end region 158 at the distal end 124 of the cage. The proximal end region 150 and the distal end region 158 are the same as shown in FIG. 6A. The second intermediate region 154 has cells 150e that are the same as the cells 150e shown in FIG. 6A. The first intermediate region 152 has a plurality of cells 132b and 132c. Cells 132c are much larger than the other cells in the cage 100 shown in FIG. 6B. Cells 132c act as skiving cells, where a central portion 134b of the cell wall 134 is weaker than at least the distal portion 134c of the cell wall 134. The distal portion 134c is stronger than the central portion 134b because of the configuration of the smaller cells 132b, which increase strength near at least the distal portion 134c of the cell wall 134 of cell 132c. Thus, the central portion deforms radially inward in response to a radially applied force to a greater extent than the distal portion. In some embodiments, the deformation of the central portion is at least about 25% more than the deformation of the distal portion. In at least some embodiments, the deformation of the central portion is at least about 30% more than the deformation of the distal portion.

In FIG. 6C, the cage 100 has a proximal end region 150 at the proximal end 122 of the cage, a first intermediate region 152, a second intermediate region 154, and a distal end region 158 at the distal end 124 of the cage. The proximal end region 150, the second intermediate region 154, and the distal end region 158 are the same as shown in FIG. 6B. The first intermediate region 152 has a plurality of cells 132b, 132c, and 132d. Cells 132c have a proximal strut pair 137, a distal strut pair 138, and a divider strut 160 that connects a strut 136 of the proximal strut pair 137 with a strut 136 of the distal strut pair. Cells 132d are axially adjacent to cells 132c. Cells 132d are much larger than the other cells in the cage 100 shown in FIG. 6B. Cells 132d act as skiving cells, where a central portion 134b of the cell wall 134 is weaker than at least the distal portion 134c of the cell wall 134. The distal portion 134c is stronger than the central portion 134b because of the configuration of the surrounding cells 132c, 132d, which increase strength near at least the distal portion 134c of the cell wall 134 of cell 132d. Thus, the central portion deforms radially inward in response to a radially applied force to a greater extent than the distal portion. In some embodiments, the deformation of the central portion is at least about 25% more than the deformation of the distal portion. In at least some embodiments, the deformation of the central portion is at least about 30% more than the deformation of the distal portion.

Many of the cells 132b in the first intermediate region 152 in FIG. 6B have been replaced in FIG. 6C by larger cells 132c in the first intermediate region 154. By having larger cells in those areas, cage 100 can be more flexible in those areas and the openings 120 created by cells 132 in the wall of the cage 100 can be positioned more favorably for removal of the clot from the wall.

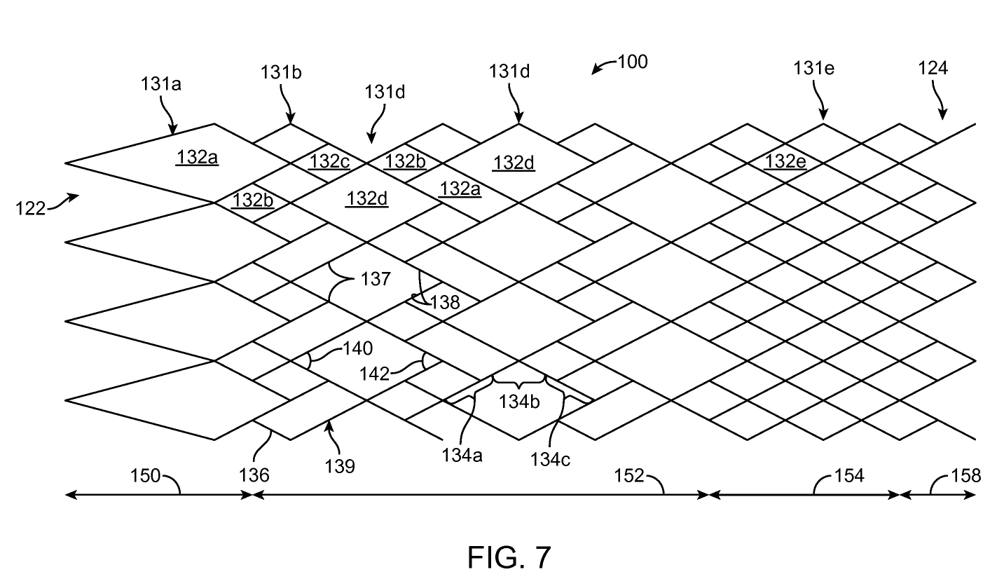

FIG. 7 shows a flat view of another embodiment of the cage 100 having a proximal end region 150 at the proximal end 122 of the cage, a first intermediate region 152, a second intermediate region 154, and a distal end region 158 at the distal end 124 of the cage.

The proximal end region 150 has a circumferential band 131a of cells 132a. Each cell 132a has a proximal strut pair 137 and a distal strut pair 138. The struts 136 of the proximal wall 137 are longer than the struts 136 of the distal wall 138.

The first intermediate region 152 has a plurality of cells 132b, 132c, 132d. Cells 132c alternate with a pair of cells 132b around a circumference of the cage in a circumferential band 131b. Cell 132b has strut pairs 137, 138 with struts 136 of equal length. Cell 132c has a proximal strut pair 137 with struts 136 that are unequal in length and a distal strut pair 138 with struts 136 that are also unequal in length. Cell 132c is the same size as two of the cells 132b. First intermediate region 152 also has a circumferential band of cells 132d that are axially adjacent to cells 132b and 132c. Cells 132d act as skiving cells, where a central portion 134b of the cell wall 134 is weaker than at least the distal portion 134c of the cell wall 134. The distal portion 134c is stronger than the central portion 134b because of the configuration of the surrounding cells 132b, 132c, which increase strength near at least the distal portion 134c of the cell wall 134 of cell 132d. Thus, the central portion deforms radially inward in response to a radially applied force to a greater extent than the distal portion. In some embodiments, the deformation of the central portion is at least about 25% more than the deformation of the distal portion. In at least some embodiments, the deformation of the central portion is at least about 30% more than the deformation of the distal portion.

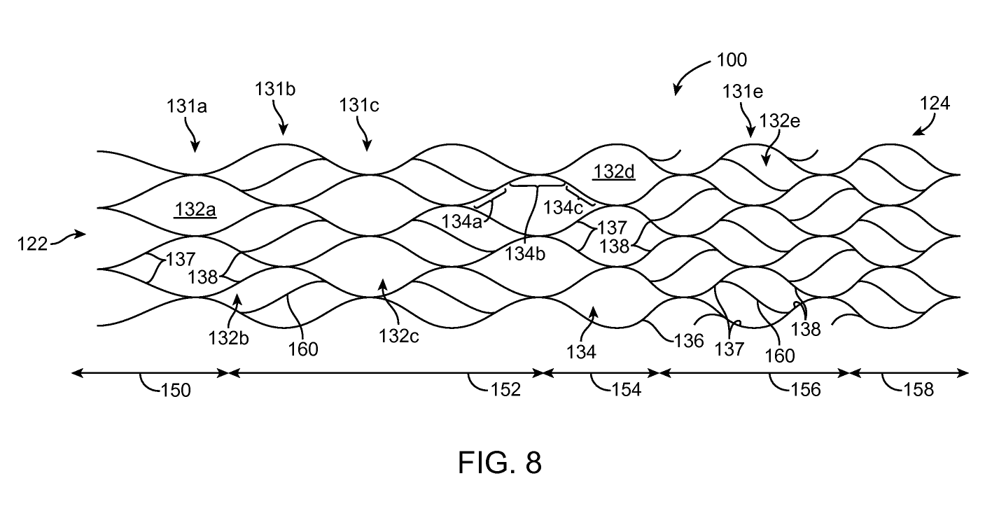

FIG. 8 shows a flat view of another embodiment of the cage 100 having a proximal end region 150 at the proximal end 122 of the cage, a first intermediate region 152, a second intermediate region 154, a third intermediate region 156, and a distal end region 158 at the distal end 124 of the cage.

The proximal end region 150 has at least one circumferential band 131a of first cells 132a having a proximal strut pair 137 and a distal strut pair 138.

The first intermediate region 152 has alternating circumferential bands of cells 132b and 132c. Cells 132b have a proximal strut pair 137, a distal strut pair 138, and a divider strut 160 that connects a strut 136 of the proximal strut pair 137 with a strut 136 of the distal strut pair. Cells 132c have a proximal strut pair 137 and a distal strut pair 138. Cells 132c act as skiving cells where a central portion 134b of the cell wall 134 is weaker than at least the distal portion 134c of the cell wall 134. The distal portion 134c is stronger than the central portion 134b because of the configuration of the surrounding cells 132c, which increase strength near at least the distal portion 134c of the cell wall 134 of cell 132b. Thus, the central portion deforms radially inward in response to a radially applied force to a greater extent than the distal portion. In some embodiments, the deformation of the central portion is at least about 25% more than the deformation of the distal portion. In at least some embodiments, the deformation of the central portion is at least about 30% more than the deformation of the distal portion.

A second intermediate region 154 has a circumferential band 131d of cells 132d having a proximal strut pair 137 with struts 136 of a longer length than struts 136 of distal strut pair 138. The cells can also act as skiving cells where a central portion 134b of the cell wall 134 is weaker than at least the distal portion 134c of the cell wall 134. Thus, the central portion deforms radially inward in response to a radially applied force to a greater extent than the distal portion.

The third intermediate region 156 has a plurality of circumferential bands 131e of cells 132e having a proximal strut pair 137, a distal strut pair 138, and a divider strut 160 that connects a strut 136 of the proximal strut pair 137 with a strut 136 of the distal strut pair 138. These smaller cells and increased density in the cells near the distal end 124 of the cage 100 allows the cage 100 to retain clot particles within the cage 100.

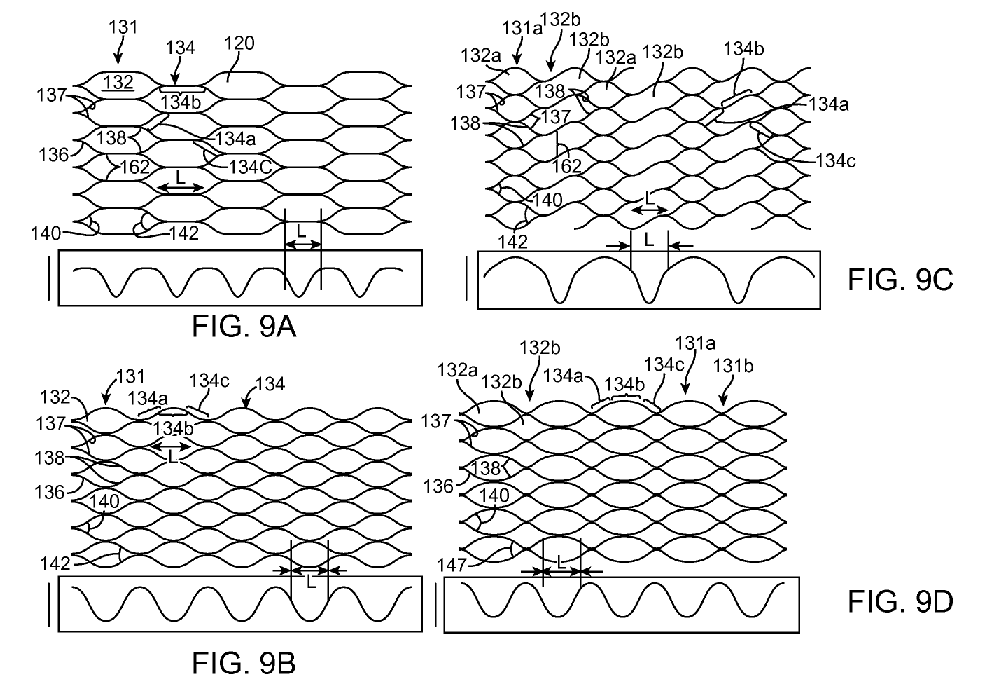

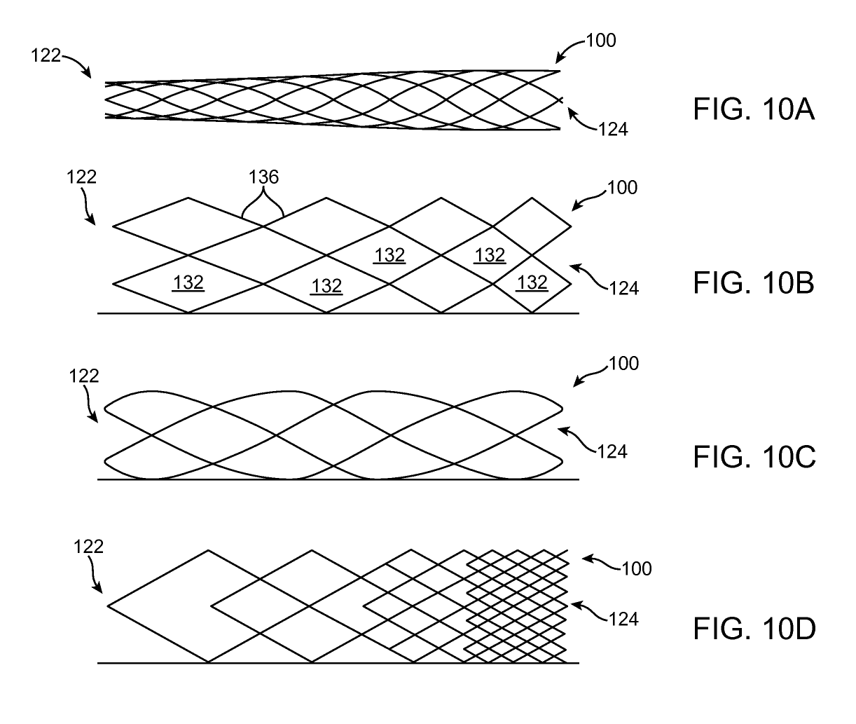

FIGS. 9A-9D show additional embodiments of cell patterns that may be used in the intermediate regions of the cage, where a skiving cell is desired. These cell patterns are shown along with graphs of the radial force along the cell pattern. As previously discussed, in some embodiments, the axial length L of the low radial force portion of the cell is at least about 0.5D, where D is the diameter of the vessel to be treated. In some embodiments, L is at least about 0.75D. In some embodiments L is about 1.0D. In some embodiments, L is between about 0.5D and about 3.0D. In some embodiments, these cell patterns can be used in at least first intermediate section 152, shown in FIG. 10. In some embodiments, these cell patterns can be used in any of the intermediate sections 152, 154, 156. In some embodiments, these cell patterns can be used in the proximal region 150 and the distal region 158.

FIG. 9A shows cells 132 with a proximal strut pair 137, a distal strut pair 138, and a central strut pair 162. The proximal strut pair 137 and the distal strut pair 138 have a wishbone shape, while the central strut pair 162 has a straight configuration. As shown in the graph below the cell pattern, the cells 132 have a local maximum radial force at the proximal and distal strut pairs 137, 138, and a local minimum radial force in the middle of the central strut pair 162. Thus, the central portion can deform radially inward in response to a radially applied force to a greater extent than at least the distal portion. While FIG. 9A shows cells of uniform construction within the cell pattern, in one embodiment of the cage 100, the pattern shown in FIG. 9A will be used to replace the cell pattern of first intermediate section 152 (and possibly second intermediate section 154) shown in FIG. 8, for example. Also, in at least one embodiment, while the cell size and shape is uniform in the pattern shown in FIG. 9A, the width, thickness, and other material properties can be varied among the cells to achieve a desired profile for the cage when expanded.

FIG. 9B has cells 132 with a proximal strut pair 137 and a distal strut pair 138. The proximal strut pair 137 and the distal strut pair 138 each have a wishbone shape. The proximal strut pair 137 and the distal strut pair 138 are thicker towards the ends of the cell 132 (in other words, the portions nearest the proximal apex angle 140 and the distal apex angle 142) than they are in the center portion of the cell 132. As shown, these cells have a local maximum radial force at the thickest regions of the cell, and a local minimum radial force in the relatively thin regions of the cell. While FIG. 9B shows cells of uniform construction within the cell pattern, in one embodiment of the cage 100, the pattern shown in FIG. 9B will be used to replace the cell pattern of first intermediate section 152 (and possibly second intermediate section 154) shown in FIG. 8.

FIG. 9C has cells 132a and cells 132b of different geometries. Cells 132a have a proximal strut pair 137 and a distal strut pair 138. Cells 132b have a proximal strut pair 137, a distal strut pair 138, and a central strut pair 162. As shown, cells 132a form a region with a relative maximum radial force, while the larger cells 132b form a region with a relative minimum radial force. A local minimum radial force occurs in the center of the cells 132b. While FIG. 9C shows cells of uniform construction within the cell pattern, in one embodiment of the cage 100, the pattern shown in FIG. 9C will be used to replace the cell pattern of first intermediate section 152 (and possibly second intermediate section 154) shown in FIG. 8.

FIG. 9D has cells 132a that are more oval-shaped than the cells 132a shown in FIG. 9A. Cells 132a have a proximal strut pair 137 and a distal strut pair 138. In this configuration, cells 132a are the skiving cells. As shown, the cells 132a have a local maximum radial force at the proximal and distal walls of the cell, and a local minimum radial force in the center of the central portion the cell. Thus, the central portion can deform radially inward in response to a radially applied force to a greater extent than at least the distal portion.

In at least one embodiment, upon full expansion, cage 100 has generally constant diameter along at least a portion of the length of the cage. In other embodiments, it may be desirable to have a cage with a tapered diameter from a proximal end to a distal end (or at least a portion thereof) or conversely the cage 100 has a tapered diameter from the distal end to the proximal end (or at least a portion thereof), as shown in FIG. 10A, upon full expansion of the cage. Various methods can be used to create a cage with a tapered diameter. By way of non-limiting example, a tapered diameter of the cage can be accomplished by progressively shortening the lengths of the struts 136 of each cell 132 along the length of the cage from the proximal end 122 to the distal end 124 (as shown in FIG. 10B), by progressively increasing the width or thickness of the struts 136 of each cell 132 along the length of the cage from the proximal end 122 to the distal end 124 (as shown in FIG. 10C), by progressively increasing cell density (in other words, the number of cells 132 per area) along the length of the cage from the proximal end 122 to the distal end 124 (as shown in FIG. 10D), or by other suitable methods.

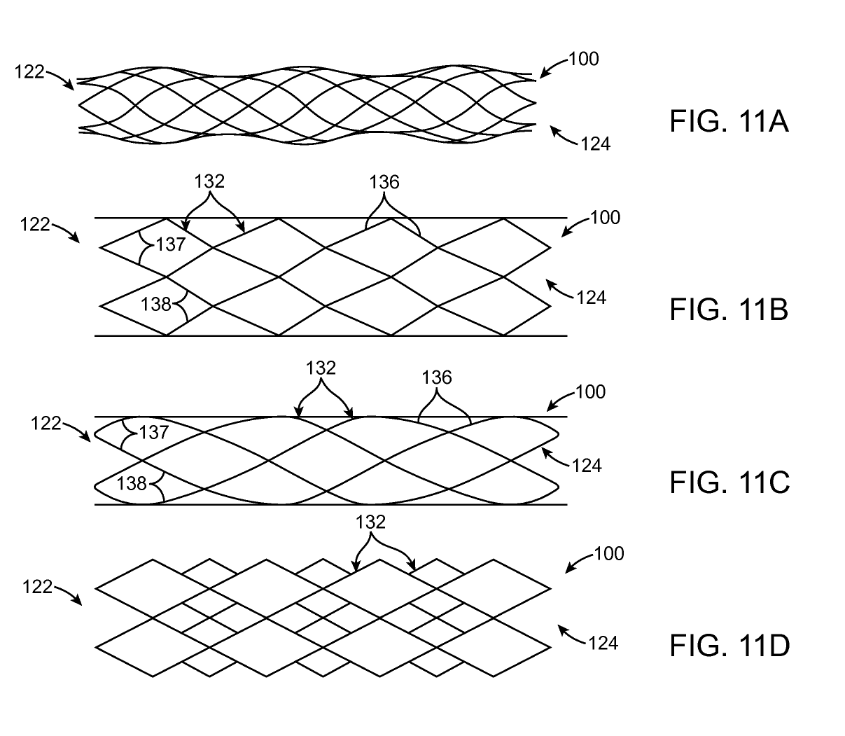

In some embodiments, it may be desirable to have a cage with a variable diameter from the proximal end to a distal end upon full expansion, such that the diameter increases and decreases repetitively along at least a portion of the length of the cage 100, as shown in FIG. 11A. Such a cage 100 can be accomplished by having proximal strut pair 137 with struts 136 of a length that is longer than the length of the struts 136 of the distal strut pair 138 (as shown in FIG. 11B), by having distal strut pair 138 with thicker or wider struts 136 of a length that is longer than the length of the struts 136 of the proximal strut pair 137 (as shown in FIG. 11C), by increasing the number of cells 132 (or increasing the cellular density) in the locations where a smaller diameter is desired (as shown in FIG. 11D), or by other suitable methods. Other configurations of the cage (such as tapered diameters in some portions of the cage and variable diameters elsewhere along the cage and other combinations) are within the scope of this invention.

In some embodiments, the cage may be provided with a distally mounted catchment or net. In such embodiments, the proximal section of the net should be a high radial pressure region to ensure the net opens up to the greatest extent of the vessel lumen as possible.

In some embodiments, the wall of the cage 100 is formed of a structural material that is present everywhere along the wall in a single layer between the proximal end and the distal end. In at least one embodiment, the cage 100 is cut from a solid tube comprised of metals, polymers, composites and other materials, such as nitinol, PET, PTFE, and other biocompatible materials. The cage can also be of a molded or other non-wire construction. In some embodiments, the wall of the cage can be formed by braiding a wire of material such as nitinol, PET, PTFE and other biocompatible materials about a mandrel.

In some embodiments, the cage is fully or partially coated on any surface of the cage with a substance, including but not limited to a drug, genetic material, cells, a therapeutic agent, a polymer matrix having a therapeutic component, a thrombolytic substance used to dissolve the clot, or any other substance which would desirable to deliver into a body lumen. The therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein, which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below (e.g. claim 3 may be taken as alternatively dependent from claim 2; claim 4 may be taken as alternatively dependent on claim 2, or on claim 3; claim 6 may be taken as alternatively dependent from claim 5; etc.).

What is claimed is:

1. A device for removing a blood clot from a lumen of a vessel, comprising:
a pusher; and
an expandable tubular cage fixedly engaged to the pusher, the tubular cage having a proximal end, a distal end, and a wall extending therebetween, the wall comprising a plurality of bands of cells axially arranged along the tubular cage, wherein one band of cells comprises at least one skiving cell having a cell wall with a proximal portion, a distal portion, and a central portion between the proximal portion and the distal portion,
wherein the central portion deforms radially inward in response to a radially applied force to a greater extent than the distal portion.

2. The device of claim 1, wherein said deformation of the central portion is at least about 25% more than said deformation of the distal portion.

3. The device of claim 2, said deformation of the central portion is at least about 30% more than said deformation of the distal portion.

4. The device of claim 1, wherein the distal portion is stiffer than at least the central portion.

5. The device of claim 1, wherein the distal portion is thicker than at least the central portion.

6. The device of claim 1, wherein the distal portion is wider than at least the central portion.

7. The device of claim 4, wherein a distal angle of the distal portion is greater than a proximal angle of the proximal portion.

8. The device of claim 4, wherein the proximal portion and the distal portion are thinner than the central portion.

9. The device of claim 1, wherein the wall is formed of a structural material arranged in a single layer such that there are no material crossover points anywhere along the wall.

10. A device for removing a blood clot from a vessel wall, comprising:
a pusher; and
an expandable tubular cage fixedly engaged to the pusher, the tubular cage having a proximal end, a distal end, and a wall extending therebetween, the wall formed of a plurality of cells defining openings in the wall of the cage, the wall comprising a proximal end region at the proximal end of the cage, a distal end region at the distal end of the cage, and at least one intermediate region therebetween,
wherein at least one of the cells of the intermediate region is a skiving cell having a cell wall with a proximal portion, a distal portion, and a central portion between the proximal portion and the distal portion, and
wherein the central portion deforms radially inward in response to a radially applied force to a greater extent than the distal portion

11. The device of claim 10, wherein said deformation of the central portion is at least about 25% more than the deformation of the distal portion.

12. The device of claim 10, the deformation of the central portion is at least about 30% more than the deformation of the distal portion.

13. The device of claim 10, wherein the at least one intermediate region has a first band of skiving cells defining first openings and a second band of cells defining second openings, the first openings being greater than the second openings.

14. The device of claim 10, wherein the intermediate region comprises at least one circumferential band of skiving cells having cell walls defined by a proximal strut pair and a distal strut pair, and an adjacent circumferential band of cells having a proximal strut pair, a distal strut pair, and a divider strut connects a first strut of the proximal strut pair to a second strut of the distal strut pair.

15. The device of claim 10, wherein a first intermediate region has at least one band of skiving cells and an axially adjacent circumferential band of cells having a greater cellular density than the band of skiving cells.

16. The device of claim 10, wherein a first intermediate region has at least one band of skiving cells and a second intermediate region has a plurality of bands of cells, wherein a cellular density of the second intermediate region is greater than a cellular density of the first intermediate region.

17. The device of claim 10, wherein the cell wall of the skiving cell comprises a proximal strut pair, a central strut pair, and a distal strut pair.
